(19) United States
(12) Patent Application Publication
(10) Pub. No.: US 2021/0282445 A1
(43) Pub. Date: Sep. 16, 2021

(54) PROTEIN ESSENTIAL AMINO ACIDS FORMULATIONS FOR HUMAN NUTRITION

(71) Applicants: Eric Jamie Wan, Hinsdale, IL (US); Jason Wan, Hinsdale, IL (US); Yang Chen, Hinsdale, IL (US)

(72) Inventors: Eric Jamie Wan, Hinsdale, IL (US); Jason Wan, Hinsdale, IL (US); Yang Chen, Hinsdale, IL (US)

(21) Appl. No.: 16/816,178

(22) Filed: Mar. 11, 2020

(51) Int. Cl. A23L 33/175; C07K 14/435; C07K 14/415; A61K 9/00; A23L 33/00; A23L 33/18; A23L 33/185; A23L 33/19

(52) U.S. Cl. CPC A23L 33/175; C07K 14/435; C07K 14/415; A61K 9/0053; A23V 2002/00; A23L 33/18; A23L 33/185; A23L 33/19; A23L 33/40

(57) ABSTRACT

This invention covers protein essential amino acids formulations as dietary supplements, nutritional formulations and foods that provide balanced and adequate levels of essential amino acid compositions to meet the recommended nutritional requirements for humans of different age groups and special need groups.

### CROSS-REFERENCE TO RELATED APPLICATIONS

This application claims the benefit of the U.S. Provisional Application No. 62/817,469, filed Mar. 12, 2019, the disclosure of which is incorporated herein by reference.

### BACKGROUND OF THE INVENTION

Healthy eating with sufficient and balanced key nutrients in diet is important for supporting normal metabolism, growth and other physiological functions in humans. Numerous dietary supplement products focusing on providing vitamins and minerals requirements to humans are commercially available in the market place. Also, fish oil capsules for providing omega-3 fatty acids to humans are commercially available. However, no formulation or commercially available products are available to provide a balanced and adequate levels of essential amino acids that are required for human nutrition.

Essential amino acids, also called indispensable amino acids, are those that have the carbon skeletons that can not be synthesized to meet body needs from simpler molecules in animal or human, and therefore must be provided in the diet. The following nine amino acids are classified as essential amino acids that humans must obtain from their diet: Histidine, Isoleucine, Leucine, Lysine, Methionine+Cysteine, Phenylalanine+Tyrosine, Threonine, Tryptophan, Valine (World Health Organization 2007⁽¹⁾, WHO Technical Report Series 935—Protein and Amino Acid Requirements in Human Nutrition).

Based on numerous and well-established scientific studies, the United States Institute of Medicine published Dietary Reference Intake values for each of the nine essential amino acids for humans of different physiological conditions, including different growth stages, age groups, gender, as well as for women during pregnancy and lactation (Institute of Medicine 2006⁽²⁾, Dietary Reference Intakes: The Essential Guide to Nutrient Requirements, The National Academies Press).

### SUMMARY OF THE INVENTION

This invention covers protein essential amino acids formulations as dietary supplements, nutritional formulations and foods, that contain balanced and adequate levels of essential amino acid compositions to meet the recommended Dietary Reference Intakes requirements for humans of different physiological conditions, including, but not limited to, different growth stages, age groups, gender, as well as for women during pregnancy and lactation.

With the current trend of consumer demand for healthier and fresher foods, particularly for those reducing consumption of traditional protein-rich diet, and favoring the fresh vegetables and fruits where protein content and essential amino acids supplies are often limited, the supplementation of balanced and adequate levels of essential amino acids in diet for this group of consumers is more beneficial. Balanced and adequate levels of essential amino acids supply in diet is particularly important for young children growth and development, pregnant women nutrition and fetus development, and lactating women nutrition and breastmilk production.

### DESCRIPTION OF THE INVENTION

This invention generally relates to protein essential amino acids formulations as dietary supplements, nutritional formulations and foods that meet the Recommended Daily Allowances (RDA) dietary requirements for essential amino acids. The RDAs of the nine essential amino acids are recommended by the Institute of Medicine for humans of different physiological conditions, including different growth stages, age groups, gender, as well as women during pregnancy and lactation. Table 1 summarizes the RDAs, extracted from the Institute of Medicine publication (Institute of Medicine 2006⁽²⁾, Dietary Reference Intakes: The Essential Guide to Nutrient Requirements, The National Academies Press).

Table 1. Recommended Daily Allowances (RDAs) of essential amino acids for humans of different growth stages, age groups, gender, as well as women during pregnancy and lactation (extracted from Institute of Medicine 2006⁽²⁾, Dietary Reference Intakes: The Essential Guide to Nutrient Requirements, The National Academies Press). The unit (mg/kg/d) represents the amount (in milligrams) of each essential amino acid, per kilogram of body weight (BW), per day.

TABLE 1

| Essential | | | | 9-13 | 9-13 | 14-18 | 14-18 | | | |
|---|---|---|---|---|---|---|---|---|---|---|
| Amino Acid | 7-12 | 1-3 | 4-8 | y/o | y/o | y/o | y/o | | pregnant | lactation |
| (mg/kg/d) | m/o | y/o | y/o | boy | girl | boy | girl | adult | women | women |
| Histidine | 32 | 21 | 16 | 17 | 15 | 15 | 14 | 14 | 18 | 19 |
| Isoleucine | 43 | 28 | 22 | 22 | 21 | 21 | 19 | 19 | 25 | 30 |
| Leucine | 93 | 63 | 49 | 49 | 47 | 47 | 44 | 42 | 56 | 62 |
| Lysine | 89 | 58 | 46 | 46 | 43 | 43 | 40 | 38 | 51 | 52 |
| Methionine + | 43 | 28 | 22 | 22 | 21 | 21 | 19 | 19 | 25 | 26 |
| cysteine | | | | | | | | | | |
| Phenylalanine + | 84 | 54 | 41 | 41 | 38 | 38 | 35 | 33 | 44 | 51 |
| tyrosine | | | | | | | | | | |
| Threonine | 49 | 32 | 24 | 24 | 22 | 22 | 21 | 20 | 26 | 30 |
| Tryptophan | 13 | 8 | 6 | 6 | 6 | 6 | 5 | 5 | 7 | 9 |
| Valine | 58 | 37 | 28 | 28 | 27 | 27 | 24 | 24 | 31 | 35 |

In this invention, the protein essential amino acids formulations processes include, but not limited to, taking into consideration of average/representative body weights (BVV) of each age group of consumers, and designing an average daily serving size to provide 50%-150% of RDA for each of the nine essential amino acids for the respective consumer group as listed in Table 1.

In this invention, the ingredients for the protein essential amino acids formulations include, but not limited to, food-grade proteins of animal (such as milk and dairy products, meat, fish and seafood, egg, animal by-products, etc.) or plant (such as legumes, soy, beans, peas, grains, wheat, oat, corn, barley, rice, nuts and seeds, peanuts, tree nuts, fruits, vegetables, etc.) origin, hydrolysates of these food-grade proteins, combinations of these food-grade proteins and/or protein hydrolysates, food-grade peptides, and food-grade pure individual essential amino acids.

In this invention, the protein essential amino acids formulations processes include, but not limited to, determination of concentrations of each essential amino acid naturally existed in the proteins, protein hydrolysates, peptides, and/or amino acids ingredients, and their combinations or mixtures.

In this invention, the protein essential amino acids formulations processes include, but not limited to, supplementation of proteins, protein hydrolysates, peptides, and combinations or mixtures of them, with selected commercially available food-grade pure essential amino acids, when needed, at required concentrations to meet the respective RDA requirements.

In this invention, the protein essential amino acids formulations may also include other food-grade additives including but not limited to flavors, colorants, texture/functionality enhancers, other nutritional ingredients such as vitamins, minerals, essential fatty acids, antioxidants, and other dietary supplements.

In this invention, the protein essential amino acids formulations processes include, but not limited to, the use of appropriate food processing technologies and processes to ensure the homogeneity of essential amino acid compositions in the formulations, and the stability of the resulting formulations.

In this invention, the protein essential amino acids formulations are, but not limited to, in dry, semi-dry, powder, solid, semi-solid, liquid, or semi-liquid forms, and may be packaged in, but not limited to, bulk, satchels, tablets, capsules, bottles, portion packages, etc. for convenience to consumers.

### DESCRIPTION OF EXAMPLES AND TABLES

Representative examples of the Protein Essential Amino Acids (ProEAA) formulations, and percentage of Recommended Daily Allowance (RDA) per serving size for each respective group of consumers are listed below. These examples serve as representative examples, and are not meant to be exhaustive of the protein essential amino acids formulations covered by this invention.

Example 1. Protein Essential Amino Acids (ProEAA) formulations containing balanced and adequate levels of essential amino acids for children of 1-3 years old

TABLE 2

| | 1-3 y/o | | ProEAA-1 | 1 y/o | 2 y/o | 3 y/o |
|---|---|---|---|---|---|---|
| | children | ProEAA-1 | formulation | BW 9.5 kg | BW12 kg | BW14 kg |
| Essential | RDA | formulation | per serve | % RDA | % RDA | % RDA |
| Amino Acids | mg/kg/d | mg/g | mg | Per serve | Per serve | Per serve |
| Histidine | 21 | 36.0 | 216 | 108 | 86 | 73 |
| Isoleucine | 28 | 53.9 | 324 | 122 | 96 | 83 |
| Leucine | 63 | 107.9 | 647 | 108 | 86 | 73 |
| Lysine | 58 | 99.3 | 596 | 108 | 86 | 73 |
| Methionine + | 28 | 47.9 | 288 | 108 | 86 | 73 |
| cysteine | | | | | | |
| Phenylalanine + | 54 | 92.5 | 555 | 108 | 86 | 73 |
| tyrosine | | | | | | |
| Threonine | 32 | 55.7 | 334 | 110 | 87 | 75 |
| Tryptophan | 8 | 16.3 | 98 | 128 | 102 | 87 |
| Valine | 37 | 63.4 | 380 | 108 | 86 | 73 |

Example 2. Protein Essential Amino Acids (ProEAA) formulations containing balanced and adequate levels of essential amino acids for children of 4-8 years old (Table 3)

TABLE 3

| | 4-8 y/o | | ProEAA-4 | 4 y/o | 6 y/o | 8 y/o |
|---|---|---|---|---|---|---|
| | children | ProEAA-4 | formulation | BW 15 kg | BW20 kg | BW26 kg |
| Essential | RDA | formulation | per serve | % RDA | % RDA | % RDA |
| Amino Acids | mg/kg/d | mg/g | mg | Per serve | Per serve | Per serve |
| Histidine | 16 | 34.8 | 278 | 116 | 87 | 67 |
| Isoleucine | 22 | 54.7 | 438 | 133 | 100 | 77 |
| Leucine | 49 | 106.4 | 851 | 116 | 87 | 67 |
| Lysine | 46 | 99.9 | 799 | 116 | 87 | 67 |
| Methionine + | 22 | 47.8 | 382 | 116 | 87 | 67 |
| cysteine | | | | | | |
| Phenylalanine + | 41 | 89.1 | 712 | 116 | 87 | 67 |
| tyrosine | | | | | | |
| Threonine | 24 | 56.5 | 452 | 125 | 94 | 72 |
| Tryptophan | 6 | 16.5 | 132 | 147 | 110 | 85 |
| Valine | 28 | 60.8 | 487 | 116 | 87 | 67 |

Example 3. Protein Essential Amino Acids (ProEAA) formulations containing balanced and adequate levels of essential amino acids for 9-13 years old boys (Table 4)

TABLE 4

| | 9-13 y/o | | ProEAA-9B | 9 y/o | 11 y/o | 13 y/o |
|---|---|---|---|---|---|---|
| | Boys | ProEAA-9B | formulation | BW 29 kg | BW36 kg | BW45 kg |
| Essential | RDA | formulation | per serve | % RDA | % RDA | % RDA |
| Amino Acids | mg/kg/d | mg/g | mg | Per serve | Per serve | Per serve |
| Histidine | 17 | 36.8 | 553 | 112 | 90 | 72 |
| Isoleucine | 22 | 54.6 | 819 | 128 | 103 | 83 |
| Leucine | 49 | 106.2 | 1593 | 112 | 90 | 72 |
| Lysine | 46 | 99.7 | 1495 | 112 | 90 | 72 |
| Methionine + | 22 | 47.7 | 715 | 112 | 90 | 72 |
| cysteine | | | | | | |
| Phenylalanine + | 41 | 88.9 | 1333 | 112 | 90 | 72 |
| tyrosine | | | | | | |
| Threonine | 24 | 56.4 | 845 | 121 | 98 | 78 |
| Tryptophan | 6 | 16.5 | 247 | 142 | 114 | 92 |
| Valine | 28 | 60.7 | 910 | 112 | 90 | 72 |

Example 4. Protein Essential Amino Acids (ProEAA) formulations containing balanced and adequate levels of essential amino acids for 9-13 years old girls (Table 5)

TABLE 5

| | 9-13 y/o | | ProEAA-9G | 9 y/o | 11 y/o | 13 y/o |
|---|---|---|---|---|---|---|
| | Girls | ProEAA-9G | formulation | BW 29 kg | BW36 kg | BW45 kg |
| Essential | RDA | formulation | per serve | % RDA | % RDA | % RDA |
| Amino Acids | mg/kg/d | mg/g | mg | Per serve | Per serve | Per serve |
| Histidine | 15 | 33.4 | 501 | 115 | 93 | 74 |
| Isoleucine | 21 | 56.1 | 841 | 138 | 111 | 89 |
| Leucine | 47 | 104.6 | 1569 | 115 | 93 | 74 |
| Lysine | 43 | 95.7 | 1435 | 115 | 93 | 74 |
| Methionine + | 21 | 46.7 | 701 | 115 | 93 | 74 |
| cysteine | | | | | | |
| Phenylalanine + | 38 | 84.6 | 1268 | 115 | 93 | 74 |
| tyrosine | | | | | | |
| Threonine | 22 | 57.9 | 868 | 136 | 110 | 88 |
| Tryptophan | 6 | 16.9 | 254 | 146 | 117 | 94 |
| Valine | 27 | 60.1 | 901 | 115 | 93 | 74 |

Example 5. Protein Essential Amino Acids (ProEAA) formulations containing balanced and adequate levels of essential amino acids for 14-18 years old boys (Table 6)

TABLE 6

| | 14-18 y/o | | ProEAA-14B | 14 y/o | 16 y/o | 18 y/o |
|---|---|---|---|---|---|---|
| | Boys | ProEAA-14B | formulation | BW 50 kg | BW60 kg | BW70 kg |
| Essential | RDA | formulation | per serve | % RDA | % RDA | % RDA |
| Amino Acids | mg/kg/d | mg/g | mg | Per serve | Per serve | Per serve |
| Histidine | 15 | 33.4 | 668 | 89 | 74 | 64 |
| Isoleucine | 21 | 56.1 | 1121 | 107 | 89 | 76 |
| Leucine | 47 | 104.6 | 2092 | 89 | 74 | 64 |
| Lysine | 43 | 95.7 | 1914 | 89 | 74 | 64 |
| Methionine + | 21 | 46.7 | 935 | 89 | 74 | 64 |
| cysteine | | | | | | |
| Phenylalanine + | 38 | 84.6 | 1691 | 89 | 74 | 64 |
| tyrosine | | | | | | |
| Threonine | 22 | 57.9 | 1157 | 105 | 88 | 75 |
| Tryptophan | 6 | 16.9 | 338 | 113 | 94 | 81 |
| Valine | 27 | 60.1 | 1202 | 89 | 74 | 64 |

Example 6. Protein Essential Amino Acids (ProEAA) formulations containing balanced and adequate levels of essential amino acids for 14-18 years old girls (Table 7)

TABLE 7

| | 14-18 y/o | | ProEAA-14G | 14 y/o | 16 y/o | 18 y/o |
|---|---|---|---|---|---|---|
| | Girls | ProEAA-14G | formulation | BW 50 kg | BW60 kg | BW70 kg |
| Essential | RDA | formulation | per serve | % RDA | % RDA | % RDA |
| Amino Acids | mg/kg/d | mg/g | mg | Per serve | Per serve | Per serve |
| Histidine | 14 | 32.2 | 645 | 92 | 77 | 66 |
| Isoleucine | 19 | 58.0 | 1160 | 122 | 102 | 87 |
| Leucine | 44 | 101.3 | 2026 | 92 | 77 | 66 |
| Lysine | 40 | 92.1 | 1842 | 92 | 77 | 66 |
| Methionine + | 19 | 43.7 | 875 | 92 | 77 | 66 |
| cysteine | | | | | | |
| Phenylalanine + | 35 | 80.6 | 1611 | 92 | 77 | 66 |
| tyrosine | | | | | | |
| Threonine | 21 | 59.9 | 1197 | 114 | 95 | 81 |
| Tryptophan | 5 | 17.5 | 350 | 140 | 117 | 100 |
| Valine | 24 | 55.2 | 1105 | 92 | 77 | 66 |

Example 7. Protein Essential Amino Acids (ProEAA) formulations containing balanced and adequate levels of essential amino acids for adult of 19 years old and over (Table 8)

TABLE 8

| | | | ProEAA-A | Adult | Adult | Adult |
|---|---|---|---|---|---|---|
| | Adult | ProEAA-A | formulation | BW 50 kg | BW60 kg | BW70 kg |
| Essential | RDA | formulation | per serve | % RDA | % RDA | % RDA |
| Amino Acids | mg/kg/d | mg/g | mg | Per serve | Per serve | Per serve |
| Histidine | 14 | 32.7 | 654 | 93 | 78 | 67 |
| Isoleucine | 19 | 58.8 | 1176 | 124 | 103 | 88 |
| Leucine | 42 | 98.0 | 1961 | 93 | 78 | 67 |
| Lysine | 38 | 88.7 | 1774 | 93 | 78 | 67 |
| Methionine + | 19 | 44.4 | 887 | 93 | 78 | 67 |
| cysteine | | | | | | |
| Phenylalanine + | 33 | 77.0 | 1541 | 93 | 78 | 67 |
| tyrosine | | | | | | |
| Threonine | 20 | 60.7 | 1214 | 121 | 101 | 87 |
| Tryptophan | 5 | 17.7 | 355 | 142 | 118 | 101 |
| Valine | 24 | 56.0 | 1120 | 93 | 78 | 67 |

Example 8. Protein Essential Amino Acids (ProEAA) formulations containing balanced and adequate levels of essential amino acids for pregnant women (Table 9)

TABLE 9

| | Pregnant | | ProEAA-PW | P women | P women | P women |
|---|---|---|---|---|---|---|
| | women | ProEAA-PW | formulation | BW 50 kg | BW60 kg | BW70 kg |
| Essential | RDA | formulation | per serve | % RDA | % RDA | % RDA |
| Amino Acids | mg/kg/d | mg/g | mg | Per serve | Per serve | Per serve |
| Histidine | 18 | 37.2 | 931 | 103 | 86 | 74 |
| Isoleucine | 25 | 52.1 | 1303 | 104 | 87 | 74 |
| Leucine | 56 | 115.8 | 2896 | 103 | 86 | 74 |
| Lysine | 51 | 105.5 | 2638 | 103 | 86 | 74 |
| Methionine + | 25 | 51.7 | 1293 | 103 | 86 | 74 |
| cysteine | | | | | | |
| Phenylalanine + | 44 | 91.0 | 2276 | 103 | 86 | 74 |
| tyrosine | | | | | | |
| Threonine | 26 | 53.8 | 1345 | 103 | 86 | 74 |
| Tryptophan | 7 | 15.7 | 393 | 112 | 94 | 80 |
| Valine | 31 | 64.1 | 1603 | 103 | 86 | 74 |

Example 9. Protein Essential Amino Acids (ProEAA) formulations containing balanced and adequate levels of essential amino acids for lactation women (Table 10)

TABLE 10

| | Lactation | | ProEAA-LW | L women | L women | L women |
|---|---|---|---|---|---|---|
| | women | ProEAA-LW | formulation | BW 50 kg | BW60 kg | BW70 kg |
| Essential | RDA | formulation | per serve | % RDA | % RDA | % RDA |
| Amino Acids | mg/kg/d | mg/g | mg | Per serve | Per serve | Per serve |
| Histidine | 19 | 33.5 | 1004 | 106 | 88 | 75 |
| Isoleucine | 30 | 55.5 | 1664 | 111 | 92 | 79 |
| Leucine | 62 | 109.2 | 3275 | 106 | 88 | 75 |
| Lysine | 52 | 91.5 | 2746 | 106 | 88 | 75 |
| Methionine + | 26 | 45.8 | 1373 | 106 | 88 | 75 |
| cysteine | | | | | | |
| Phenylalanine + | 51 | 89.8 | 2694 | 106 | 88 | 75 |
| tyrosine | | | | | | |
| Threonine | 30 | 57.2 | 1717 | 114 | 95 | 82 |
| Tryptophan | 9 | 16.7 | 502 | 112 | 93 | 80 |
| Valine | 35 | 61.6 | 1849 | 106 | 88 | 75 |

What are claimed are:

1. Essential amino acids formulations as dietary supplements, nutritional formulations and foods, that contain balanced and adequate levels of essential amino acid compositions to meet the recommended Dietary Reference Intakes requirements for humans of different physiological conditions, including, but not limited to, different growth stages, age groups, gender, as well as for women during pregnancy and lactation.

2. The essential amino acids formulations of claim 1, further comprising processes including, but not limited to, taking into consideration of average/representative body weights (BVV) of each age group of consumers, and designing an average daily serving size to provide 50%-150% of RDA for each of the nine essential amino acids for the respective consumer group.

3. The essential amino acids formulations of claim 1, further comprising ingredients including, but not limited to, food-grade proteins of animal (such as milk and dairy products, meat, fish and seafood, egg, animal by-products, etc.) or plant (such as legumes, soy, beans, peas, grains, wheat, oat, corn, barley, rice, nuts and seeds, peanuts, tree nuts, fruits, vegetables, etc.) origin, hydrolysates of these food-grade proteins, combinations of these food-grade proteins and/or protein hydrolysates, food-grade peptides, and food-grade pure individual essential amino acids.

4. The essential amino acids formulations of claim 1, further comprising processes including, but not limited to, determination of concentrations of each essential amino acid naturally existed in the proteins, protein hydrolysates, peptides, and/or amino acids ingredients, and their combinations or mixtures.

5. The essential amino acids formulations of claim 1, further comprising processes including, but not limited to, supplementation of proteins, protein hydrolysates, peptides, and combinations or mixtures of them, with selected commercially available food-grade pure essential amino acids, when needed, at required concentrations to meet the respective RDA requirements.

6. The essential amino acids formulations of claim 1, further comprising food-grade additives/ingredients including but not limited to flavors, colorants, texture/functionality enhancers, other nutritional ingredients such as vitamins, minerals, essential fatty acids, antioxidants, and other dietary supplements.

7. The essential amino acids formulations of claim 1, further comprising processes including, but not limited to, the use of appropriate food processing technologies and processes to ensure the homogeneity of essential amino acid compositions in the formulations, and the stability of the resulting formulations.

8. The essential amino acids formulations of claim 1, further comprising formulations in, but not limited to, dry, semi-dry, powder, solid, semi-solid, liquid, or semi-liquid forms, and may be packaged in, but not limited to, bulk, satchels, tablets, capsules, bottles, portion packages, etc. for convenience to consumers.

9. The essential amino acids formulations of claim 1, further comprising formulations containing balanced and adequate levels of essential amino acids for children of 1-3 years old.

10. The essential amino acids formulations of claim 1, further comprising formulations containing balanced and adequate levels of essential amino acids for children of 4-8 years old.

11. The essential amino acids formulations of claim 1, further comprising formulations containing balanced and adequate levels of essential amino acids for 9-13 years old boys.

12. The essential amino acids formulations of claim 1, further comprising formulations containing balanced and adequate levels of essential amino acids for 9-13 years old girls.

13. The essential amino acids formulations of claim 1, further comprising formulations containing balanced and adequate levels of essential amino acids for 14-18 years old boys.

14. The essential amino acids formulations of claim 1, further comprising formulations containing balanced and adequate levels of essential amino acids for 14-18 years old girls.

15. The essential amino acids formulations of claim 1, further comprising formulations containing balanced and adequate levels of essential amino acids for adult of 19 years old and over.

16. The essential amino acids formulations of claim 1, further comprising formulations containing balanced and adequate levels of essential amino acids for pregnant women.

17. The essential amino acids formulations of claim 1, further comprising formulations containing balanced and adequate levels of essential amino acids for lactation women.
